(19) United States
(12) Patent Application Publication
(10) Pub. No.: US 2013/0102640 A1
(43) Pub. Date: Apr. 25, 2013

(54) METHOD FOR THE PREPARATION OF CIS-1,2-DIOLS IN THE KILOGRAM SCALE

(71) Applicants: Werner Mederski, Zwingenberg (DE); Georg Stoehr, Fischbachtal (DE); Andreas Werner, Hainburg (DE)

(72) Inventors: Werner Mederski, Zwingenberg (DE); Georg Stoehr, Fischbachtal (DE); Andreas Werner, Hainburg (DE)

(73) Assignee: MERCK PATENT GESELLSCHAFT MIT BESCHRANKTER HAFTUNG, Darmstadt (DE)

(21) Appl. No.: 13/807,796

(22) PCT Filed: Jun. 3, 2011
(86) PCT No.: PCT/EP2011/002741

§ 371 Date: Dec. 31, 2012

(30) Foreign Application Priority Data
Jul. 1, 2010 (EP) 10006796.6

(51) Int. Cl. C07D 209/48; A61K 45/06; C07B 41/02; A61K 31/4035

(52) U.S. Cl. CPC C07D 209/48; A61K 31/4035; A61K 45/06; C07B 41/02
USPC 514/352; 548/473; 546/310; 514/417

(57) ABSTRACT

The present invention relates to the scale up of the preparation of cis-1,2-diols of formula I

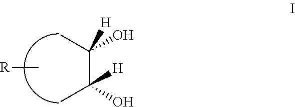

from the gram to the kilogram scale.

### FIELD OF THE INVENTION

The present invention relates to the scale up of the preparation of cis-1,2-diols of formula I to the kilogram scale.

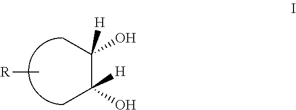

The present invention preferably relates to the scale up of the preparation of the compound N-((1R,2S,3R)-2,3-Dihydroxy-cyclohexyl)-3-(2-fluoro-4-iodo-phenylamino)-isonicotinamide to the kilogram scale.

### BACKGROUND OF THE INVENTION

The background of the present invention covers the dihydroxlation of olefins, also termed as alkenes, into cis-1,2-diols.

Originally, the olefins were reacted with osmium tetroxide, which is highly toxic, volatile and difficult to handle. Although there are also catalytic variants (so-called Upjohn process based on the publication: VanRheenen, V. et al., Tet. Lett. 1976, 23, 1973-1976) and less-volatile reagents (or example K₂OsO₂(OH)₄) there is a risk of contamination of the product, which is unacceptable for substances which are used as medicaments for human use.

A significant improvement was provided by the method according to B. Plietker, which uses RuO₄ formed in situ from RuCl₃ with addition of the reoxidant NalO₄ and either a Brönsted acid H₂SO₄ or a Lewis acid CeCl₃. The possible mechanism is discussed in Plietker B., et al., Chem. 2004, 2, 1116-1124 and Plietker B. and M. Niggemann, J. Org. Chem., 2005, 70, 2402-2405. According to the Plietker process, the dihydroxylation mixture consisting of RuCl₃, NalO₄ and CeCl₃ is initially introduced in a solvent mixture (ethyl acetate, acetonitrile and water in the ratio 3:3:1) and cooled and the olefin is then added in one portion. Furthermore, the reaction is very fast, highly exothermic and difficult to control and therefore critical with respect to safety, reproducibility, yield and purity. Finally, the side products have to be costly removed.

In attempting to apply this method to the preparation of N-((1R,2S,3R)-2,3-Dihydroxy-cyclohexyl)-3-(2-fluoro-4-iodo-phenylamino)-isonicotinamide and to scale up from the millimol to the mole scale, the limits of feasibility are quickly reached. The exothermicity is less controllable, the larger the batch. Temperatures of 40° C. or more are reached, especially critical with respect to the flammable solvent mixture. This results in a drastic loss of yield and the glycol cleavage of the side reaction dominates. For example, in experiments in a 1 g batch only 60% of product was obtained, in a 100 g batch only 30%. The yield and purity were very low even when cooled to −10° C. with acetone ice trying to control the exothermicity. Accordingly, the Plietker method is not applicable for the large-scale preparation of cis-1,2-diols. Thus, the preparation methods of the prior art provide products with, for pharmaceutical applications unacceptable contaminations or result in drastic losses of yield and uncontrollable exothermicity.

The aim of the present invention was to provide a method for the preparation of cis-1,2-diols, which are especially used for pharmaceutical purposes, in the kilogram scale without the described disadvantages.

### BRIEF DESCRIPTION OF THE INVENTION

Embodiment of the present invention is a method for the preparation of cis-1,2-diols of formula I in the kilogram scale,

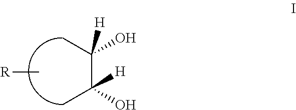

wherein the ring system is a 4- to 8-membered cycloalkyl and R represents one residue selected from the group consisting of hydrogen, alkyl, alkoxy, O-acetyl, carbonyl, alkoxycarbonyl, cyano, halogen, isoindole-1,3-dionyl, tert-butoxycaboxyaminyl bis-(tert-butoxycarboxy)aminyl and a residue according to formula II or formula III, wherein

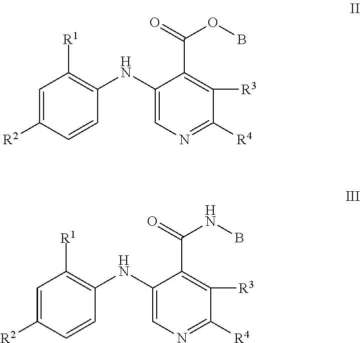

B is a compound according to formula I
R¹ is hydrogen, methyl, ethyl, n-propyl, i-propyl, SH or Hal,
R² is hydrogen, methoxy, ethoxy, acetylene, cyano, SH or Hal,
R³, R⁴ are independently selected from hydrogen, SH or Hal and

### Hal is F, Cl, Br or I,

characterized in that a compound according to formula IV,

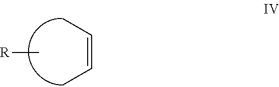

wherein the ring system is a 4- to 8-membered cycloalkyl and R is as defined above, is prelayed in a solvent mixture in an inert atmosphere and a dihydroxylation mixture is added and the reaction container is cooled.

Another embodiment of the present invention is the method according to the present invention, characterized in that the cis-1,2-diol is a compound of formula V,

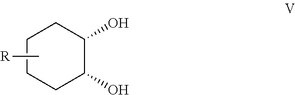

wherein R is as defined above.

A further embodiment of the present invention is the method according to the invention, characterized in that the cis-1,2-diol is 2-((1R,2S,3R)-2,3-Dihydroxy-cyclohexyl)-isoindole-1,3-dione (see compound of formula VI).

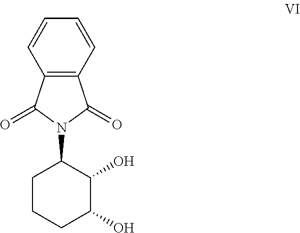 [SMILES: O=C1c2ccccc2C(=O)N1[C@@H]1CCC[C@@H](O)[C@H]1O]

A preferred embodiment of the present invention is the method according to the invention, characterized in that the cis-1,2-diol is a compound of formula VII,

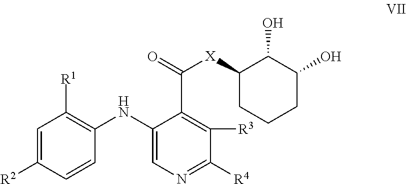

wherein

### X is NH or O,

R¹ is hydrogen, methyl, ethyl, n-propyl, i-propyl, SH or Hal,
R² is hydrogen, methoxy, ethoxy, acetylene, cyano, SH or Hal,
R³, R⁴ are independently selected from hydrogen, SH or Hal and

### Hal is F, Cl, Br or I;

or a pharmaceutically acceptable salt, stereoisomer or tautomer thereof, including mixtures thereof in all ratios.

A particularly preferred embodiment of the present invention is the method of the present invention, characterized in that the cis-1,2-diol or the compound of formula VII is a compound selected form the group consisting of N-((1R,2S,3R)-2,3-Dihydroxy-cyclohexyl)-3-(2-fluoro-4-iodo-phenylamino)-isonicotinamide (see compound of formula VIII), 2-Chloro-N-((1R,2S,3R)-2,3-dihydroxy-cyclohexyl)-5-(2-fluoro-4-iodo-phenylamino)-isonicotinamide (see compound of formula IX), N-((1R,2S,3R)-2,3-Dihydroxy-cyclohexyl)-3-(2-fluoro-4-iodo-phenylamino)-isonicotinamide (see compound of formula X), 3-(4-Bromo-2-fluoro-phenylamino)-N-((1R,2S,3R)-2,3-dihydroxy-cyclohexyl)-isonicotinamide (see compound of formula XI) and N-((1R,2S,3R)-2,3-Dihydroxy-cyclohexyl)-3-(2-fluoro-phenylamino)-isonicotinamide (see compound of formula XII), or a pharmaceutically acceptable salt, stereoisomer or tautomer thereof, including mixtures thereof in all ratios.

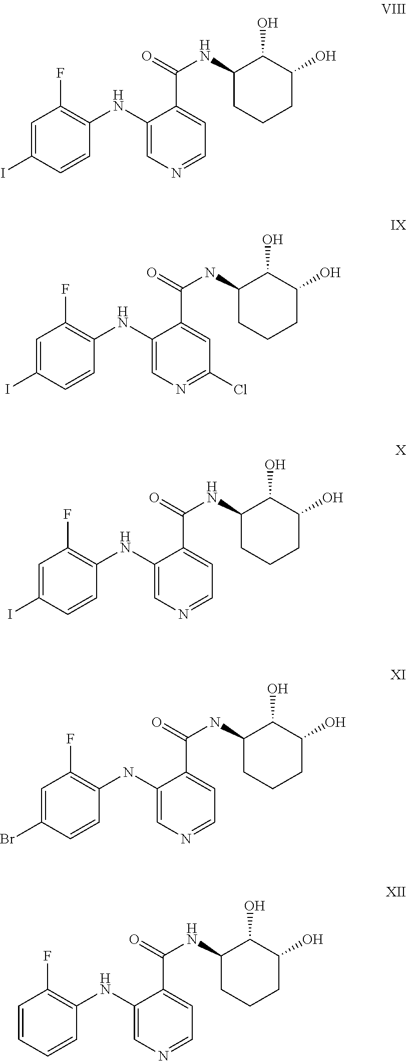 [SMILES: O=C(N[C@@H]1CCC[C@@H](O)[C@H]1O)c1ccncc1Nc1ccc(I)cc1F ; O=C(N[C@@H]1CCC[C@@H](O)[C@H]1O)c1cc(Cl)ncc1Nc1ccc(I)cc1F ; O=C(N[C@@H]1CCC[C@@H](O)[C@H]1O)c1ccncc1Nc1ccc(I)cc1F ; O=C(N[C@@H]1CCC[C@@H](O)[C@H]1O)c1ccncc1Nc1ccc(Br)cc1F ; O=C(N[C@@H]1CCC[C@@H](O)[C@H]1O)c1ccncc1Nc1ccccc1F]

A further embodiment of the present invention are compounds of the formulae I, V, VI, VII, VIII, IX, X, XI, XII or a pharmaceutically acceptable salt, stereoisomer or tautomer thereof, including mixtures thereof in all ratios, prepared by a preparation method according to the present invention.

The method of the present invention is an efficient method for the dihydroxylation of cyclic olefins on a kilogram scale. The method of the invention surprisingly solves all problems and critical disadvantages of the prior art techniques being easily controllable especially with respect to the exothermicity and moreover the reaction can be always stopped or interrupted leading to higher safety and reproducibility. Furthermore, the method of the present invention provides high yields and purity and gets along without critical toxic agents and contaminations thereof leading to high safety. Especially, the method of the present invention makes it possible for the first time to provide in a safe, easy and cost-effective way cis-1,2-diols, preferably those which are used for pharmaceutical products, in the kilogram scale with a high yield and purity.

Further advantages of the method of the present invention are that side reactions are suppressed, the dihydroxylation mixture remains always in an active state and the amount of necessary reoxidation agent (e.g. NalO₄) and Lewis acid (e.g. CeCl₃) can be significantly reduced leading to lower costs and contaminations of the product. All in all, the method of the present invention is the first method for the preparation of cis-1,2-diols being unproblematic when performed in large-scale and being highly reproducible.

The present invention provides a modification of the Plietker process but instead of merely being applicable in bench-scale it surprisingly makes it possible to synthesize kilogram quantities safely and with high yields.

Surprisingly, high yield and high purity are obtained if the starting material is initially introduced in the solvent mixture in an inert atmosphere, the mixture is cooled and the pre-prepared dihydroxylation mixture is added in portions.

The yields of the method of the present invention are between 60 and 100%, preferably 70 and 90%, particularly preferred between 75 and 85%. The purity is between 90 and 100%, preferably 95 and 100%, particularly preferred between 99 and 100%.

The solvent mixture according to the present invention consists of two or more solvents selected from the group consisting of dichloromethane, chlorobenzene, tetrachloromethane, 2-methoxy-2-methylpropane, 2-isopropoxypropane, cyclohexanone, acetone, 2-methyl-pentan-2-one, 2-methyl-tetrahydrofurane, tetrahydrofurane, acetic acid ethyl ester, acetic acid propyl ester, acetic acid isopropyl etser, acetic acid isobutyl ester, acetonirile and water preferred are mixtures of 2-methyl-tetrahydrofurane, acetic acid ethyl ester, acetic acid propyl ester, acetic acid isopropyl ester, acetic acid, isobutyl ester, acetonitrile and water, preferred are mixtures of two to four of said solvents, particularly preferred are mixtures of three solvents, especially preferred is a mixture of ethyl acetate, acetonitrile and water, preferably in the ratio [2-4]: [2-4]: [0.1-2], particularly preferred in the ratio [2.5-3.5]: [2.5-3.5]: [0.4-1.2], especially in the ratio 3:3:0.6.

The mixture containing the starting material and the solvent mixture is cooled to −10° C. to 20° C., preferably to −5° C. to 10° C., particularly preferred to −1° C. to 5° C., especially to −0.5° C. to 0.5° C., the reaction container is cooled and the method of the present invention is conducted at a temperature of −10° C. to 20° C., preferably at a temperature of −5° C. to 10° C., particularly preferred at a temperature of −1° C. to 5° C., especially at a temperature of −0.5° C. to 0.5° C.

Preferably the mixture containing the starting material and the solvent mixture are exposed to an inert atmosphere, preferably to an argon or nitrogen atmosphere, particularly preferred to a nitrogen atmosphere.

The method of the present invention is based on a Lewis acid accelerated bimetallic RuCl₃/NalO₄ reoxidation system. The dihydroxylation mixture according to the present invention consists of a reoxidation agent, a catalyst and an acid. A particularly preferred dihydroxylation mixture according to the present invention is RuCl₃/CeCl₃/NalO₄

An appropriate reoxidation agent according to the present invention is NalO₄, but NalO₄ is only an example for an appropriate reoxidation agent and the present invention is not limited to this example. Accordingly, other known and appropriate reoxidation agents may be used according to the present invention.

An appropriate catalyst according to the present invention is RuCl₃, but RuCl₃ is only an example for an appropriate catalyst and the present invention is not limited to this example. Accordingly, other known and appropriate catalysts may be used according to the present invention.

Appropriate acids according to the present invention are Lewis or Brönsted acids. Appropriate Lewis acids according to the present invention are selected from the group consisting of LiCl, RbCl, FeCl₃, FeCl₂, SrCl₂, NiCl₂, CoCl₂, ZnCl₂ and CeCl₃, particularly preferred are ZnCl₂, CeCl₃, FeCl₃, and FeCl₂. According to the present invention, instead of Lewis acids, Brönsted acids may also be used. Examples of appropriate Brönsted acids according to the present invention are H₂SO₄, HCl, NH₄+ or HCO₃−. However, the disclosed Lewis and Brönsted acids are only examples of appropriate acids according to the present invention, the present invention is not limited to these examples and other known and appropriate Lewis and Brönsted acids may be used according to the present invention.

The dihydroxylation mixture is added in portions, dropwise or more rapidly in larger portions or as slow continuous inflow to the mixture containing the starting material and the solvent mixture.

The disadvantages of the method according to Plietker are the high costs of NalO₄, the problem of the disposal thereof and the contamination of the pharmaceutical product with it. According to the method of the present invention the amount of the reoxidation agent, e.g. NalO₄, can be reduced to 1.2 M, preferably to 1 M, instead of 1.5 M which are necessary according to the Plietker method, leading to advantages as lower cost of goods, lower amount of wastage and higher safety.

According to the method of the present invention the amount of the acid, e.g. the Lewis acid, e.g. CeCl₃, can be reduced to 5-15 Mol-%, preferably to 7-12 Mol-%, particularly preferred to 10 Mol-%, providing a further advantage over the Plietker method.

According to the method of the present invention the amount of the catalyst, e.g. RuCl₃, is 0.1-0.6 Mol-%, preferably 0.2-0.4 Mol-%, particularly preferred 0.25-0.3 Mol-%.

According to the invention, the term “scale up” means the transfer of the reaction conditions for the production of the cis-1,2-diols according to the invention taken out of the laboratory to the manufacture pipeline in industry, i.e. from the gram to the kilogram scale.

The synthesis of cis-1,2-diols of formula I, exemplified by the preparation of the MEK inhibitor N-((1R,2S,3R)-2,3-Dihydroxy-cyclohexyl)-3-(2-fluoro-4-iodo-phenylamino)-isonicotinamide, is achieved according to the following reaction scheme:

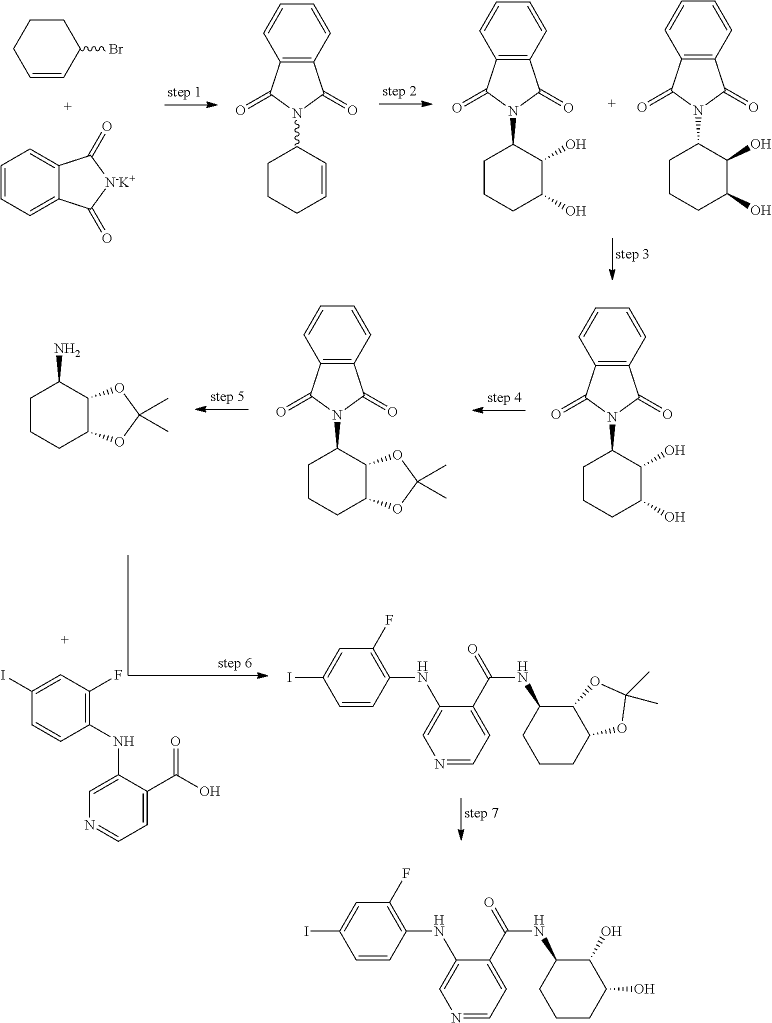

Thus, according to the above scheme the present invention concerns the process optimization of reaction step 2, where the educt of formula XIII prelayed in a solvent mixture is converted to 2-((1R,2S,3R)-2,3-Dihydroxy-cyclohexyl)-isoindole-1,3-dione (compound of formula VI) by cis-hydroxylation by adding a dihydroxylation mixture consisting of RuCl₃, NalO₄ and CeCl₃.

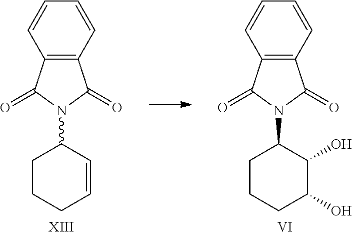

Every reaction step concerning the synthesis of cis-1,2-diols can optionally be followed by one or more working up and/or isolating procedures. Suitable procedures are known in the art, for example from standard works, such as Houben-Weyl, Methoden der organischen Chemie [Methods of Organic Chemistry], Georg-Thieme-Verlag, Stuttgart). Examples for such procedures include, but are not limited to evaporating a solvent, distilling, crystallization, fractionised crystallization, extraction procedures, washing procedures, digesting procedures, filtration procedures, chromatography, for example by HPLC, and drying procedures, especially drying procedures in vacuum and/or elevated temperature.

The final product according to above exemplified reaction scheme is known as N-((1R,2S,3R)-2,3-Dihydroxy-cyclohexyl)-3-(2-fluoro-4-iodo-phenylamino)-isonicotinamide and was filed for patent application with the USPTO under the filing No. 61/137,858. This patent application describes the phenylamino isonicotinamide compounds of formula VII as inhibitors of the Ras/Raf/MEK/ERK pathway, which is frequently referred to as the MAP kinase pathway. MAPK stands for mitogen-activated protein kinase.

The Ras/Raf/MEK/ERK pathway is a central signal transduction pathway, which transmits signals from multiple cell surface receptors to transcription factors in the nucleus which regulate gene expression. This pathway can be stimulated by mitogens, cytokines and growth factors (Steelman et al., Leukemia 2004, 18, 189-218). Depending upon the stimulus and cell type, this pathway can transmit signals, which result in the prevention or induction of apoptosis or cell cycle progression. The Ras/Raf/MEK/ERK pathway has been shown to play important roles in cell proliferation and the prevention of apoptosis. Aberrant activation of this pathway is commonly observed in malignantly transformed cells. Amplification of ras proto-oncogenes and activating mutations that lead to the expression of constitutively active Ras proteins are observed in approximately 30% of all human cancers (Stirewalt et al., Blood 2001, 97, 3589-95). Mutated, oncogenic forms of Ras are found in 50% of colon and >90% pancreatic cancers as well as many other types of cancers (Kohl et al., Science 1993, 260, 1834-1837). The effects of Ras on proliferation and tumorigenesis have been documented in immortal cell lines (McCubrey et al., Int J Oncol 1995, 7, 295 310). bRaf mutations have been identified in more than 60% of malignant melanoma (Davies, H et al., Nature 2002, 417, 949-954). Given the high level of mutations that have been detected in Ras, this pathway has always been considered a key target for therapeutic intervention (Chang et al., Leukemia 2003, 17, 1263-93).

The Ras/Raf/MEK/ERK signaling pathway can regulate proliferation through downstream transcription factor targets including NF-^{κ}B, CREB, Ets-1, AP-1 and c-Myc. ERKs can directly phosphorylate Ets-1, AP-1 and c-Myc, which lead to their activation. Alternatively, ERKs can phosphorylate and activate the downstream kinase target RSK, which then phosphorylates and activates transcription factors, such as CREB. These transcription factors induce the expression of genes important for cell cycle progression, for example, Cdk's, cyclins, growth factors, and for apoptosis prevention, for example, antiapoptotic Bcl-2 and cytokines. Overall, treatment of cells with growth factors leads to the activation of ERKs which results in proliferation and, in some cases, differentiation (Lewis et al., Adv. Cancer Res, 1998, 74, 49-139). The MEK family of genes consists of five genes: MEK1, MEK2, MEK3, MEK4 and MEK5. This family of dual-specificity kinases has both serine/threonine and tyrosine kinase activity. The structure of MEK consists of an amino-terminal negative regulatory domain and a carboxy-terminal MAP kinase-binding domain, which is necessary for binding and activation of ERKs. Deletion of the regulatory MEK1 domain results in constitutive MEK1 and ERK activation (Steelman et al., Leukemia 2004, 18, 189-218).

MEK1 is a 393-amino-acid protein with a molecular weight of 44 kDa (Crews et al., Science 1992, 258, 478-80). MEK1 is modestly expressed in embryonic development and is elevated in adult tissue with the highest levels detected in brain tissue. MEK1 requires phosphorylation of S218 and S222 for activation, and substitution of these residues with either aspartic acid (D) or glutamic acid (E) led to an increase in activity and foci formation in NIH3T3 cells (Huang et al., Mol Biol Cell, 1995, 6, 237-45). Constitutive activity of MEK1 in primary cell culture promotes senescence and induces p53 and p16^{INK4a}, and the opposite was observed in immortalized cells or cells lacking either p53 or p16^{INK4a} (Lin et al., Genes Dev, 1998, 12, 3008-3019). Constitutive activity of MEK1 inhibits F-κ^{κ}B transcription by negatively regulating p38 MAPK activity (Carter et al., J Biol Chem 2000, 275, 27858-64). The main physiological substrates of MEK are the members of the ERK (extracellular signal-regulated kinase) or MAPK (mitogen activated protein kinase) family of proteins. Aberrant expression of MEK1 has been detected in many different types of cancer, and mutated forms of MEK1 will transform fibroblast, hematopoietic and other cell types.

Constitutive activation of MEK1 results in cellular transformation. MEK1 therefore represents a likely target for pharmacological intervention in proliferative and inflammatory diseases (Lee et al., Nature 1994, 372, 739-746; Dudley et al., Proc. Natl. Acad. Sci. U.S.A. 1995, 92, 7686-7689).

Useful inhibitors of MEK have been developed that show potential therapeutic benefit in several studies. For example, small molecule MEK inhibitors have been shown to inhibit human tumor growth in nude mouse xenografts (Yeh, T. et al, Proceedings of the American Association of Cancer Research 2004, 45, Abs 3889 and Lee, P. et al., Proceedings of the American Association of Cancer Research 2004, 45, Abs 3890). MEK inhibitors also entered clinical trials, i.e. ARRY142886 (Wallace, E. et al, Proceedings of the American Association of Cancer Research 2004, 45, Abs 3891; Adjei, A. A. et al, Journal of Clinical Oncology 2008, 26, 2139-2146; Shannon, A. M. et al, Molecular Cancer Therapeutics 2007, 6, 3414S-3415S Part 2), PD-0325901 (Swanton C, Johnston S IDDB MEETING REPORT 2003, February 13-1; Haura, E. B. et al, Molecular Cancer Therapeutics 2007, 6, 3468S-3469S Part 2; LoRusso, P. A. et al, Molecular Cancer Therapeutics 2007, 6, 3469S-3470S Part 2), PD-184352 (Waterhouse et al., Proceedings of the American Society for Clinical Oncology 2003, 22, Abs 816), XL-518 (Johnston, S., Molecular Cancer Therapeutics 2007, 6, 3595S-3595S Part 2), RDEA-119 (2007 press release), and RDEA-436 (2008 press release).

The term “pharmaceutically acceptable salts” of the compounds prepared by the method according to the invention refers to conventional non-toxic salts and includes acid addition salts such as organic acid salts (e.g. acetate, trifluoroacetate, maleate, tartrate, methanesulfonate, benzenesulfonate, formate, toluenesulfonate), inorganic acid salt (e.g. hydrochloride, hydrobromide, hydroiodide, sulfate, nitrate, phosphate), or salts with an amino acid (e.g. arginine, aspartic acid, glutamic acid), or metal salts such as alkali metal salts (e.g. sodium salt, potassium salt) and alkaline earth metal salts (e.g. calcium salt, magnesium salt), ammonium salts, or organic base salts (e.g. trimethylamine salt, triethylamine salt, pyridine salt, picoline salt, dicyclohexylamine salt, N,N′-dibenzylethylenediamine salt).

As used herein, the term “solvate” preferably refers to a complex of variable stoichiometry formed by a solute and a solvent. The term solvates of the compounds is therefore taken to mean adductions of inert solvent molecules onto the compounds, which form owing to their mutual attractive force. Such solvents for the purpose of the invention may not interfere with the biological activity of the solute. Preferably, the solvent used is a pharmaceutically acceptable solvent. Examples of suitable pharmaceutically acceptable solvents include, without limitation, water, ethanol and acetic acid. Most preferably the solvent used is water. Solvates are for example monohydrates, dihydrates or alcoholates.

A physiologically acceptable salt of a compound prepared by the method according to the invention can also be obtained by isolating and/or treating the according to the present invention obtained by the described reaction with an acid or a base.

A base of a compound prepared by the method according to the invention can be converted into the associated acid-addition salt using an acid, for example by reaction of equivalent amounts of the base and the acid in a preferably inert solvent, such as ethanol, followed by evaporation. Suitable acids for this reaction are, in particular, those which give physiologically acceptable salts. Thus, it is possible to use inorganic acids, for example sulfuric acid, sulfurous acid, dithionic acid, nitric acid, hydrohalic acids, such as hydrochloric acid or hydrobromic acid, phosphoric acids, such as, for example, orthophosphoric acid, sulfamic acid, furthermore organic acids, in particular aliphatic, alicyclic, araliphatic, aromatic or heterocyclic monobasic or polybasic carboxylic, sulfonic or sulfuric acids, for example formic acid, acetic acid, propionic acid, hexanoic acid, octanoic acid, decanoic acid, hexadecanoic acid, octadecanoic acid, pivalic acid, diethylacetic acid, malonic acid, succinic acid, pimelic acid, fumaric acid, maleic acid, lactic acid, tartaric acid, malic acid, citric acid, gluconic acid, ascorbic acid, nicotinic acid, isonicotinic acid, methane- or ethanesulfonic acid, ethanedisulfonic acid, 2-hydroxyethanesulfonic acid, benzenesulfonic acid, trimethoxybenzoic acid, adamantanecarboxylic acid, p-toluenesulfonic acid, glycolic acid, embonic acid, chlorophenoxyacetic acid, aspartic acid, glutamic acid, proline, glyoxylic acid, palmitic acid, parachlorophenoxyisobutyric acid, cyclohexanecarboxylic acid, glucose 1-phosphate, naphthalenemono- and -disulfonic acids or laurylsulfuric acid.

Salts with physiologically unacceptable acids, for example picrates, can be used to isolate and/or purify the compound according to the present invention.

On the other hand, a compound prepared by the method according to the invention can be converted into the corresponding metal salts, in particular alkali metal salts or alkaline earth metal salts, or into the corresponding ammonium salts, using bases (for example sodium hydroxide, potassium hydroxide, sodium carbonate or potassium carbonate). Suitable salts are furthermore substituted ammonium salts, for example the dimethyl-, diethyl- and diisopropylammonium salts, monoethanol-, diethanol- and diisopropanolammonium salts, cyclohexyl- and dicyclohexylammonium salts, dibenzylethylenediammonium salts, furthermore, for example, salts with arginine or lysine.

If desired, the free bases of the compounds of the present invention can be liberated from their salts by treatment with strong bases, such as sodium hydroxide, potassium hydroxide, sodium carbonate or potassium carbonate, so long as no further acidic groups are present in the molecule. In the cases where the compounds of the present invention have free acid groups, salt formation can likewise be achieved by treatment with bases. Suitable bases are alkali metal hydroxides, alkaline earth metal hydroxides or organic bases in the form of primary, secondary or tertiary amines.

The compounds of the present invention can be in the form of a prodrug compound. The prodrug form is also within of the scope of the preset invention. “Prodrug compound” means a derivative that is converted into a biologically active compound according to the present invention under physiological conditions in the living body, e.g. by oxidation, reduction, hydrolysis or the like, each of which is carried out enzymatically, or without enzyme involvement. Examples of prodrugs are compounds, wherein the amino group in a compound of the present invention is acylated, alkylated or phosphorylated, e.g. eicosanoylamino, alanylamino, pivaloyloxymethylamino or wherein the hydroxyl group is acylated, alkylated, phosphorylated or converted into the borate, e.g. acetyloxy, palmitoyloxy, pivaloyloxy, succinyloxy, fumaryloxy, alanyloxy or wherein the carboxyl group is esterified or amidated, or wherein a sulfhydryl group forms a disulfide bridge with a carrier molecule, e.g. a peptide, that delivers the drug selectively to a target and/or to the cytosol of a cell. These compounds can be produced from compounds of the present invention according to well-known methods. Other examples of prodrugs are compounds, wherein the carboxylate in a compound of the present invention is for example converted into an alkyl-, aryl-, choline-, amino, acyloxymethylester, linolenoyl-ester. Metabolites of compounds of the present invention are also within the scope of the present invention.

Where tautomerism, e.g., keto-enol tautomerism, of compounds of the present invention or their prodrugs may occur, the individual forms, e.g. the keto or the enol form, are claimed separately and together as mixtures in any ratio. The same applies for stereoisomers, e.g. enantiomers, isomers, conformers and the like. If desired, isomers can be separated by methods well known in the art, e.g. by liquid chromatography. The same applies for enantiomers, e.g. by using chiral stationary phases. Additionally, enantiomers may be isolated by converting them into diastereomers, i.e. coupling with an enantiomerically pure auxiliary compound, subsequent separation of the resulting diastereomers and cleavage of the auxiliary residue. Alternatively, any enantiomer of a compound of the present invention may be obtained from stereoselective synthesis using optically pure starting materials

The compounds of formula VII prepared by the method according to the invention may be used for the formulation of pharmaceutical compositions comprising a compound or a pharmaceutically acceptable salt, stereoisomer or tautomer thereof, including mixtures thereof in all ratios, as an active ingredient together with a pharmaceutically acceptable carrier.

“Pharmaceutical composition” means one or more active ingredients, and one or more inert ingredients that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions encompass any composition made by admixing a compound of formula VII prepared by the method according to the invention and a pharmaceutically acceptable carrier.

A pharmaceutical composition may additionally comprise one or more other compounds of formula VII prepared by the method according to the invention as active ingredients or other MEK inhibitors, or other pharmaceutically active agents other than the compounds of the present invention.

The pharmaceutical compositions include compositions suitable for oral, rectal, topical, parenteral (including subcutaneous, intramuscular, and intravenous), ocular (ophthalmic), pulmonary (nasal or buccal inhalation), or nasal administration, although the most suitable route in any given case will depend on the nature and severity of the conditions being treated and on the nature of the active ingredient. They may be conveniently presented in unit dosage form and prepared by any of the methods well-known in the art of pharmacy.

In one embodiment, said compounds of formula VII prepared by the method according to the invention may be used as one of above pharmaceutical compositions for the treatment of cancer such as brain, lung, squamous cell, bladder, gastric, pancreatic, breast, head, neck, renal, kidney, ovarian, prostate, colorectal, oesophageal, testicular, gynecological, thyroid cancer, melanoma, hematologic malignancies such as acute myelogenous leukemia, multiple myeloma, chronic myelogneous leukemia, myeloid cell leukemia, or any other type of solid or liquid tumors. In another embodiment, said pharmaceutical composition is for the treatment of a noncancerous hyperproliferative disorder such as benign hyperplasia of the skin (e.g., psoriasis), restenosis, polycystic kidney disease, or prostate (e.g., benign prostatic hypertrophy (BPH)) and also for the treatment of inflammatory and autoimmune diseases such as rheumatoid arthritis, Crohn's disease, asthma, ulcerative colitis, irritable bowel syndrome, multiple sclerosis, lupus erythematosus and others. In another embodiment, said pharmaceutical composition is for the treatment of genetic conditions characterized by upregulation of the MEK/ERK pathway such as the Costello syndrome, Noonan syndrome, cardiofaciocutaneous syndrome and others.

The invention also relates to the use of compounds of formula VII prepared by the method according to the invention for the preparation of a medicament for the treatment of hyperproliferative diseases related to the hyperactivity of MEK as well as diseases modulated by the MEK cascade in mammals, or disorders mediated by aberrant proliferation, such as cancer and inflammation.

The invention also relates to a compound of formula VII prepared by the method according to the invention or a pharmaceutical composition according to the invention for the treatment of pancreatitis or kidney disease (including proliferative glomerulonephritis and diabetes induced renal disease) or pain in a mammal which comprises a therapeutically effective amount of a compound of the present invention, or a pharmaceutically acceptable salt, stereoisomer or tautomer thereof and a pharmaceutically acceptable carrier.

The invention also relates to a compound of formula VII prepared by the method according to the invention or a pharmaceutical composition according to the invention for the prevention of blastocyte implantation in a mammal which comprises a therapeutically effective amount of a compound of the present invention, or a pharmaceutically acceptable salt, stereoisomer or tautomer thereof, including mixtures thereof in all ratios, and a pharmaceutically acceptable carrier.

The invention also relates to a compound of formula VII prepared by the method according to the invention or a pharmaceutical composition according to the invention for treating a disease related to vasculogenesis or angiogenesis in a mammal which comprises a therapeutically effective amount of a compound of the present invention, or a pharmaceutically acceptable salt, stereoisomer or tautomer thereof, including mixtures thereof in all ratios, and a pharmaceutically acceptable carrier.

In one embodiment, said compound of formula VII prepared by the method according to the invention or a pharmaceutical composition according to the invention is for treating a disease selected from the group consisting of tumor angiogenesis, chronic inflammatory disease such as rheumatoid arthritis, inflammatory bowel disease, atherosclerosis, skin diseases such as psoriasis, eczema, and sclerodema, diabetes, diabetic retinopathy, retinopathy of prematurity, age-related macular degeneration, hemangioma, glioma, melanoma, Kaposi's sarcoma and ovarian, breast, lung, pancreatic, prostate, colon and epidermoid cancer.

This invention also relates to a compound of formula VII prepared by the method according to the invention or a pharmaceutical composition according to the invention for inhibiting abnormal cell growth in a mammal which comprises an amount of a compound of the present invention, or a pharmaceutically acceptable salt, stereoisomer or tautomer thereof, including mixtures thereof in all ratios, in combination with an amount of another anti-cancer therapeutic selected from the group consisting of mitotic inhibitors, alkylating agents, antimetabolites, intercalating antibiotics, growth factor inhibitors, antiangiogenic agents, cell cycle inhibitors, enzyme inhibitors, topoisomerase inhibitors, biological response modifiers, antihormones, angiogenesis inhibitors, and anti-androgens, or immune modulators, wherein the amounts of the compound or salt, stereoisomer or tautomer thereof and of the chemotherapeutic are together effective in inhibiting abnormal cell growth. Many anti-cancer therapeutics are presently known in the art. In one embodiment, the anti-cancer therapeutic is a chemotherapeutic selected from the group consisting of mitotic inhibitors, alkylating agents, anti-metabolites, intercalating antibiotics, growth factor inhibitors, cell cycle inhibitors, enzymes, topoisomerase inhibitors, biological response modifiers, anti-hormones, angiogenesis inhibitors, and anti-androgens. In another embodiment the anti-cancer therapeutic is an antibody selected from the group consisting of bevacizumab, CD40-specific antibodies, chTNT-1/B, denosumab, zanolimumab, IGF1R-specific antibodies, lintuzumab, edrecolomab, WX G250, rituximab, ticilimumab, trastuzumab and cetuximab.

This invention further relates to a method for inhibiting abnormal cell growth in a mammal or treating a hyperproliferative disorder that comprises administering to the mammal an amount of a compound of formula VII prepared by the method according to the invention or a pharmaceutically acceptable salt, stereoisomer or tautomer thereof, in combination with radiation therapy, wherein the amounts of the compound, pharmaceutically acceptable salt, stereoisomer or tautomer thereof, is in combination with the radiation therapy effective in inhibiting abnormal cell growth or treating the hyperproliferative disorder in the mammal. Techniques for administering radiation therapy are known in the art, and these techniques can be used in the combination therapy described herein. The administration of a compound of the invention in this combination therapy can be determined as described herein. It is believed that the compounds of the present invention can render abnormal cells more sensitive to treatment with radiation for purposes of killing and/or inhibiting the growth of such cells.

Accordingly, this invention further relates to a method for sensitizing abnormal cells in a mammal to treatment with radiation which comprises administering to the mammal an amount of a compound of formula VII prepared by the method according to the invention or a pharmaceutically acceptable salt, stereoisomer or tautomer thereof, which amount is effective is sensitizing abnormal cells to treatment with radiation. The amount of the compound, salt, stereoisomer or tautomer in this method can be determined according to the means for ascertaining effective amounts of such compounds described herein. The invention also relates to a method for inhibiting abnormal cell growth in a mammal that comprises an amount of a compound of formula VII prepared by the method according to the invention or a pharmaceutically acceptable salt, stereoisomer, tautomer or isotopically-labeled derivative thereof, and an amount of one or more substances selected from anti-angiogenesis agents, signal transduction inhibitors, and antiproliferative agents.

In practical use, the compounds of the present invention can be combined as the active ingredient in intimate admixture with a pharmaceutical carrier according to conventional pharmaceutical compounding techniques. The carrier may take a wide variety of forms depending on the form of preparation desired for administration, e.g. oral or parenteral (including intravenous). In preparing the compositions for oral dosage form, any of the usual pharmaceutical media may be employed, such as, for example, water, glycols, oils, alcohols, flavoring agents, preservatives, coloring agents and the like. In the case of oral liquid preparations, any of the usual pharmaceutical media may be employed, such as, for example, suspensions, elixirs and solutions; or carriers such as starches, sugars, microcrystalline cellulose, diluents, granulating agents, lubricants, binders, disintegrating agents and the like. In the case of oral solid preparations the composition may take forms such as, for example, powders, hard and soft capsules and tablets, with the solid oral preparations being preferred over the liquid preparations.

Because of their ease of administration, tablets and capsules represent the most advantageous oral dosage unit form in which case solid pharmaceutical carriers are obviously employed. If desired, tablets may be coated by standard aqueous or non-aqueous techniques. Such compositions and preparations should contain at least 0.1 percent of active compound. The percentage of active compound in these compositions may, of course, be varied and may conveniently be between about 2 percent to about 60 percent of the weight of the unit. The amount of active compound in such therapeutically useful compositions is such that an effective dosage will be obtained. The active compounds can also be administered intranasally as, for example liquid drops or spray.

The tablets, pills, capsules, and the like may also contain a binder such as gum tragacanth, acacia, corn starch or gelatin; excipients such as dicalcium phosphate; a disintegrating agent such as corn starch, potato starch, alginic acid; a lubricant such as magnesium stearate; and a sweetening agent such as sucrose, lactose or saccharin. When a dosage unit form is a capsule, it may contain, in addition to materials of the above type, a liquid carrier such as a fatty oil.

Various other materials may be present as coatings or to modify the physical form of the dosage unit. For instance, tablets may be coated with shellac, sugar or both. A syrup or elixir may contain, in addition to the active ingredient, sucrose as a sweetening agent, methyl and propylparabens as preservatives, a dye and a flavoring such as cherry or orange flavor.

Compounds of the present invention may also be administered parenterally. Solutions or suspensions of these active compounds can be prepared in water suitably mixed with a surfactant such as hydroxy-propylcellulose. Dispersions can also be prepared in glycerol, liquid polyethylene glycols and mixtures thereof in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (e.g. glycerol, propylene glycol and liquid polyethylene glycol), suitable mixtures thereof, and vegetable oils.

Any suitable route of administration may be employed for providing a mammal, especially a human, with an effective dose of a compound of the present invention. For example oral, rectal, topical, parenteral, ocular, pulmonary, nasal, and the like may be employed. Dosage forms include tablets, troches, dispersions, suspensions, solutions, capsules, creams, ointments, aerosols, and the like. Preferably compounds of the present invention are administered orally.

The effective dosage of active ingredient employed may vary depending on the particular compound employed, the mode of administration, the condition being treated and the severity of the condition being treated. Such dosage may be ascertained readily by a person skilled in the art.

When treating or preventing cancer, inflammation or other proliferative diseases for which compounds of the present invention are indicated, generally satisfactory results are obtained when the compounds of the present invention are administered at a daily dosage of from about 0.01 milligram to about 100 milligram per kilogram of animal body weight, preferably given as a single daily dose. For most large mammals, the total daily dosage is from about 0.1 milligrams to about 1000 milligrams, preferably from about 0.2 milligram to about 50 milligrams. In the case of a 70 kg adult human, the total daily dose will generally be from about 0.2 milligrams to about 200 milligrams. This dosage regimen may be adjusted to provide the optimal therapeutic response.

A further embodiment of the present invention are also sets (kits) consisting of separate packets of
a.) a therapeutically effective amount of one or more compounds of formula VII prepared by the method according to the invention and
b.) a therapeutically effective amount of one or more further pharmaceutically active agents other than the compounds of the present invention.

The set comprises suitable containers, such as boxes, individual bottles, bags or ampoules. The set may, for example, comprise separate ampoules, each containing an effective amount of a compound of formula VII prepared by the method according to the invention and/or pharmaceutically acceptable salts, stereoisomers and tautomers thereof, including mixtures thereof in all ratios, and an effective amount of a further medicament active ingredient in dissolved or lyophilised form.

A further embodiment of the present invention is also a process for the preparation of a pharmaceutical composition, characterized in that one or more of the compounds of formula VII prepared by the method according to the invention and one or more compounds selected from the group consisting of solid, liquid or semiliquid excipients, auxiliaries, adjuvants, diluents, carriers and pharmaceutically active agents other than the compounds according to the invention, are converted in a suitable dosage form.

The compounds of the present invention can be prepared according to the procedures of the above and following schemes and examples, using appropriate materials. The method of the present invention is further exemplified by the following specific examples. Unless otherwise indicated in the schemes and examples, the variables have the same meaning as described above. Moreover, by utilizing the procedures described herein, in conjunction with ordinary skills in the art, additional compounds of the present invention claimed herein can be readily prepared. The compounds and methods illustrated in the examples are not, however, to be construed as forming the only genus that is considered as the invention. The examples further illustrate details for the preparation of the compounds of the present invention. Those skilled in the art will readily understand that known variations of the conditions and processes of the following preparative procedures can be used to prepare these compounds.

The instant compounds are generally isolated in the form of their pharmaceutically acceptable salts, such as those described above. The amine-free bases corresponding to the isolated salts can be generated by neutralization with a suitable base, such as aqueous sodium hydrogencarbonate, sodium carbonate, sodium hydroxide and potassium hydroxide, and extraction of the liberated amine-free base into an organic solvent, followed by evaporation. The amine-free base, isolated in this manner, can be further converted into another pharmaceutically acceptable salt by dissolution in an organic solvent, followed by addition of the appropriate acid and subsequent evaporation, precipitation or crystallization.

The detailed description with its examples should not be construed to limit the scope of the present invention.

### Example 1

### Experimental Procedure

To a dry, nitrogen-purged 400-L reactor with a mechanically stirred solution of 36.01 of acetonitrile, 36.01 of ethyl acetate and 2.01 of water are added 4545.2 g (20.0 mol) of racemic 2-cyclohex-2-enylisoindole-1,3-dione at +2° C. The pre-prepared oxidation reagent mixture [1280.0 g (6.0 mol) of sodium metaperiodate are suspended in 1400 ml of water, 186.0 g (0.37 mol) of cesium(III) chloride heptahydrate are added, and the mixture is stirred for 2 minutes. 60.0 ml (14.4 mol) of a 5% solution of ruthenium(III) chloride in water are then added, and the mixture is stirred for a further 1 minute] is then added rapidly. During this addition, the temperature rises to +4° C. The mixture is stirred at 0° C. for a further 1 hour. The procedure is repeated a further three times. The reaction temperatures rise to +4.5° C., +4.5° C. and +5.0° C. respectively. The reagent consumption is in total 5120 g (23.94 mol) of NalO₄, 744.0 g (2.0 mol) of Ce(III) Cl₃.7H₂O and 240.0 ml (57.85 mmol) of Ru(III) Cl₃/5%. The conversion increases from 20% via 42% and 60% to 80%, with less than 5% of the olefin being detected by HPLC at the end. When the addition is complete, the reaction mixture is warmed to 15° C. over the course of one hour. 200 l of ethyl acetate and 50 l of water are added successively to the suspension, which is then stirred. After separation of the organic phase, the aqueous phase is washed with 10 l of ethyl acetate. The organic phases are combined and washed three times with 50 l of water each time, subsequently twice with 20 l of dilute sodium sulfite solution each time and finally a further three times with 10 l of water each time. After 12 hours, the organic suspension is concentrated to a volume of 15 l, cooled in ice for 2 hours, filtered with suction and rinsed with 3 l of 2-methoxy-2-methylpropane, giving 4270 g (yield 82%) of rac-[(1R,2S,3R-1S,2R,3S)-2,3-dihydroxycyclohexyl]isoindole-1,3-dione as white crystals having a melting point of 221-222° C.; ¹H-NMR (8, ppm, DMSO-d₆): 7.87-7.81 (4H, m), 4.78 (1H, d, J=5.8 Hz), 4.50 (1H, d, J=2.9 Hz), 4.30 (1H, ddd, J=12.6, 10.6 and 3.9 Hz), 4.06 (1H, ddd, J=10.6, 5.8 and 2.8 Hz), 3.95 (1H, m), 2.02 (1H, m), 1.78-1.60 (3H, m), 1.49-1.39 (2H, m). MS (EI): m/e 261.

### Example 2

### Testing of Biological Activity

### 2.1 MEK-1 Enzyme Assay (LANCE-HTRF)

The activity of the compounds of the present invention may be determined by the following procedure: Inhibition of human MEK1 kinase activity was monitored with a homogenous, fluorescence based assay. The assay uses time resolved fluorescence resonance energy transfer to probe for phosphorylation of ERK1 by MEK1. The assay is carried out in low volume 96 well microtiterplates. In a total volume of 15 μl, compounds are incubated with 100 nM MEK1, 15 μM ATP, 300 nM ERK2 employing a buffer containing 20 mM TRIS/HCl, 10 mM MgCl₂, 100 μM NaVO₄, 1 mM DTT, and 0.005% Tween 20 (pH 7.4). After two hours, 5 nM Europium-anti-PY20 (Perkin Elmer) and 50 nM Anti-GST-Allophycocyanin (CisBio) in buffer containing 50 mM EDTA and 0.05% BSA are added and the reaction incubated for one hour in the dark. Time-resolved fluorescence is measured using a LJL-Analyst (Molecular Devices) with an excitation wavelength of 340 nm and an emission wavelength of 665 nm. The final concentration of DMSO is 2%. To assess the inhibitory potential of the compounds, IC50-values are determined.

### 2.2 Tumor Cell Proliferation Assays (ATP Lite)

Murine colon C26, human melanoma A375 and human pancreatic MiaPaCa-2 cells are plated in 96 well Corning white plates (1500 cells/well for C26, and 2000 cells/well for A375, and MiaPaCa-2) and cultured overnight at 37° C. in 5% CO₂. Inhibitors are serially diluted in 100% DMSO and subsequently added to cells to reach a final concentration of 0.25% DMSO. The cells are incubated for 4 days in the presence of test compounds in cell growth media (DMEM with 10% fetal bovine serum, 2 mM glutamine for C26, and MiaPaCa-2, and RPMI with 10% fetal bovine serum, 2 mM glutamine for A375). Cell proliferation is quantified using the ATP lite cell proliferation kit (Packard).

### 2.3 In Vivo Efficacy Studies (Mouse Xenograft Models)

Male nude (nu/nu) mice are injected subcutaneously above the right foreleg with certain number of cells of human tumor cell lines such as Colo-205, A375 or MiaPaCa2. Tumors are measured with calipers one week after cells are implanted. Tumor length (l) and width (w) are measured and tumor volume is calculated with the equation l*w²/2. Animals are sorted into groups so that each group has a mean tumor volume of 150-200 mm³ and treatments with compounds are started (designated as Day 0). Tumor volume and body weight are measured for each animal on Days 0, 4, 6, 8, 10, 12 and 14. Tumor volume and percent body weight are analyzed via Two-Way Repeated Measures Analysis of Variance (RM-ANOVA) followed by Fisher's post-hoc multiple pair-wise comparisons of treatment group means.

### BRIEF DESCRIPTION OF THE DRAWINGS

FIG. 1 is an HPCL chromatography of compound of formula VI

FIG. 2 is ¹H-NMR of compound of formula VI

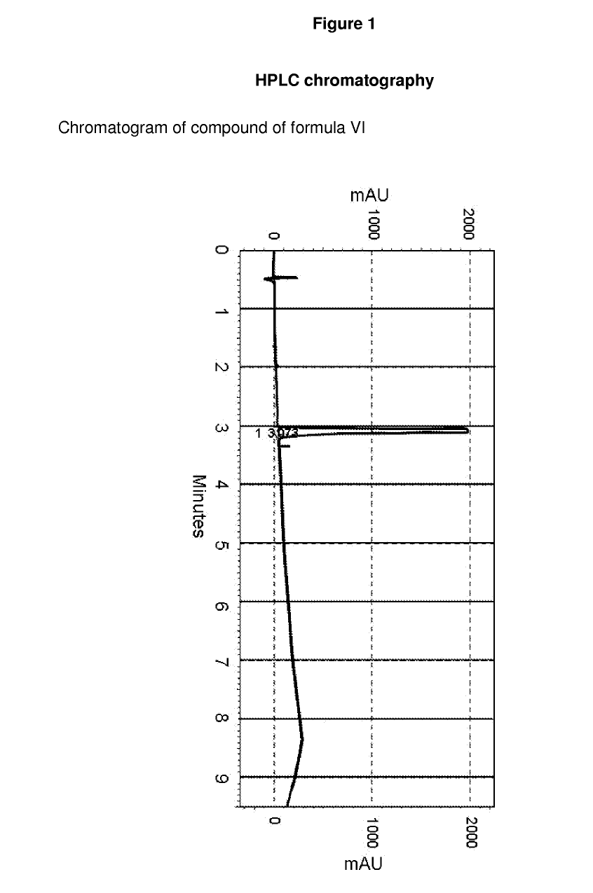

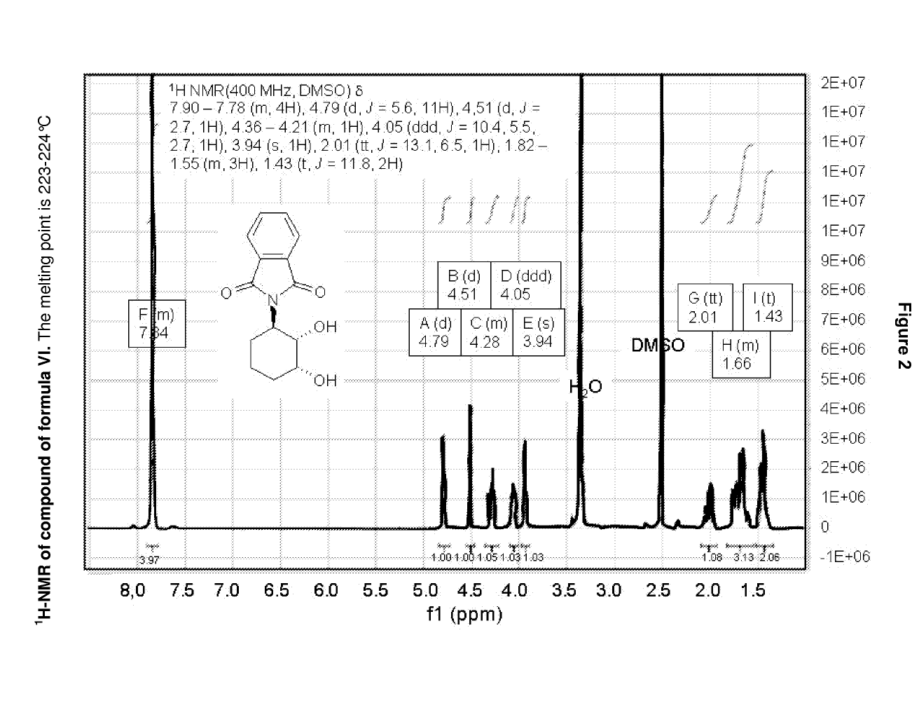

### FIG. 1: Measurement Conditions

Method: Flow: 2 mL/min
   Gradient: 95% A [symbol] 5% A
   Eluent A: Water+0.3% Trifluoroacetic acid
   Eluent B: Acetonitrile+0.3% Trifluoroacetic acid+0.1% Water
Detection: Wavelength 220 nm
Column: Chromolith SpeedROD (RP-18e, 50-4.6 mm)

1. A method for the preparation of cis-1,2-diols of formula I in the kilogram scale, 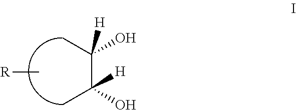
wherein the ring system is a 4- to 8-membered cycloalkyl and R represents one residue selected from the group consisting of hydrogen, alkyl, alkoxy, O-acetyl, carbonyl, alkoxycarbonyl, cyano, halogen, isoindole-1,3-dionyl, tert-butoxycaboxyaminyl bis-(tert-butoxycarboxy)aminyl and a residue according to formula II or formula III wherein 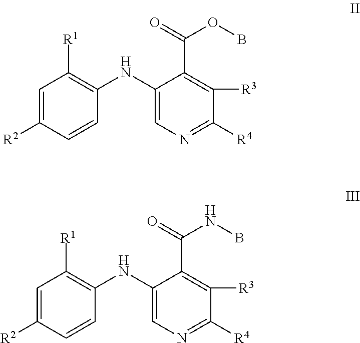
B is a compound according to formula I
R¹ is hydrogen, methyl, ethyl, n-propyl, i-propyl, SH or Hal,
R² is hydrogen, methoxy, ethoxy, acetylene, cyano, SH or Hal,
R³, R⁴ are independently selected from hydrogen, SH or Hal and
Hal is F, Cl, Br or I,
characterized in that a compound according to formula IV, 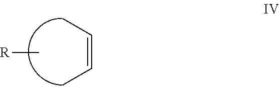
wherein the ring system is a 4- to 8-membered cycloalkyl and R is as defined above, is prelayed in a solvent mixture in an inert atmosphere and a dihydroxylation mixture is added and the reaction container is cooled.

2. A method according to claim 1, characterized in that the cis-1,2-diol is a compound of formula V, 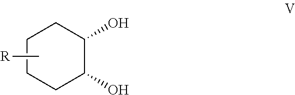
wherein R is as defined in claim 1.

3. A method according to claim 1, characterized in that the cis-1,2-diol is 2-((1R,2S,3R)-2,3-Dihydroxy-cyclohexyl)-isoindole-1,3-dione.

4. A method according to claim 1, characterized in that the cis-1,2-diol is a compound of formula VII, 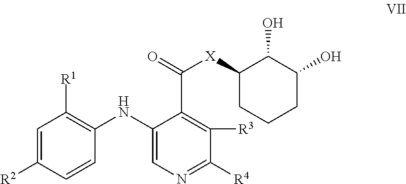
wherein
X is NH or O,
R¹ is hydrogen, methyl, ethyl, n-propyl, i-propyl, SH or Hal,
R² is hydrogen, methoxy, ethoxy, acetylene, cyano, SH or Hal,
R³, R⁴ are independently selected from hydrogen, SH or Hal and
Hal is F, Cl, Br or I;
or a pharmaceutically acceptable salt, stereoisomer or tautomer thereof, including mixtures thereof in all ratios.

5. A method according to claim 1, characterized in that the cis-1,2-diol is the is selected from the group consisting of N-((1R,2S,3R)-2,3-Dihydroxy-cyclohexyl)-3-(2-fluoro-4-iodo-phenylamino)-isonicotinamide, 2-Chloro-N-((1R,2S,3R)-2,3-dihydroxy-cyclohexyl)-5-(2-fluoro-4-iodo-phenylamino)-isonicotinamide, N-((1R,2S,3R)-2,3-Dihydroxy-cyclohexyl)-3-(2-fluoro-4-iodo-phenylamino)-isonicotinamide, 3-(4-Bromo-2-fluoro-phenylamino)-N-((1R,2S,3R)-2,3-dihydroxy-cyclohexyl)-isonicotinamide and N-((1R,2S,3R)-2,3-Dihydroxy-cyclohexyl)-3-(2-fluoro-phenylamino)-isonicotinamide or a pharmaceutically acceptable salt, stereoisomer or tautomer thereof, including mixtures thereof in all ratios.

6. A method according to claim 1, characterized in that it is conducted at a temperature of −5° to 10° C.

7. A method according to claim 6, characterized in that it is conducted at a temperature of −0.5° to 0.5° C.

8. A method according to claim 1, characterized in that the solvent mixture consists of EtOAc/CH₃CN/H₂O.

9. A method according to claim 8, characterized in the said solvent mixture is used in a ratio of 3:3:0.6.

10. A method according to claim 1, characterized in that the dihydroxylation mixture is RuCl₃/CeCl₃/NalO₄.

11. A compound of formula I, prepared by a preparation method according to claim 1.

12. A compound of formula V, prepared by a preparation method according to claim 2.

13. A compound of formula VI 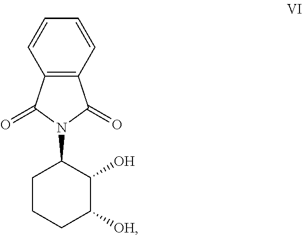 [SMILES: O=C1c2ccccc2C(=O)N1[C@@H]1CCC[C@@H](O)[C@H]1O]
prepared by a preparation method according to claim 1.

14. A compound of formula VII or a pharmaceutically acceptable salt, stereoisomer or tautomer thereof, prepared by a preparation method according to claim 4.

15. A compound according to claim 14, characterized in that the compound is selected from the group consisting of N-((1R,2S,3R)-2,3-Dihydroxy-cyclohexyl)-3-(2-fluoro-4-iodo-phenylamino)-isonicotinamide, 2-Chloro-N-((1R,2S,3R)-2,3-dihydroxy-cyclohexyl)-5-(2-fluoro-4-iodo-phenylamino)-isonicotinamide, N-((1R,2S,3R)-2,3-Dihydroxy-cyclohexyl)-3-(2-fluoro-4-iodo-phenylamino)-isonicotinamide, 3-(4-Bromo-2-fluoro-phenylamino)-N-((1R,2S,3R)-2,3-dihydroxy-cyclohexyl)-isonicotinamide and N-((1R,2S,3R)-2,3-Dihydroxy-cyclohexyl)-3-(2-fluoro-phenylamino)-isonicotinamide or a pharmaceutically acceptable salt, stereoisomer or tautomer thereof, including mixtures thereof in all ratios.

16. A method for treating and/or preventing a disease or condition which comprises administering a therapeutically effective amount of a compound of claim 14 to a patient in need thereof.

17. A method according to claim 16 wherein the disease is selected from the group consisting of cancer, inflammation, pancreatitis or kidney disease, pain, benign hyperplasia of the skin, restenosis, prostate, diseases related to vasculogenesis or angiogenesis, tumor angiogenesis, skin diseases selected from psoriasis, eczema, and sclerodema, diabetes, diabetic retinopathy, retinopathy of prematurity, age-related macular degeneration, hemangioma, glioma, melanoma and Kaposi's sarcoma.

18. Pharmaceutical composition, characterized in that it contains a therapeutically effective amount of one or more compounds according to claim 14.

19. Pharmaceutical composition according to claim 18, characterized in that it contains one or more additional compounds, selected from the group consisting of physiologically acceptable excipients, auxiliaries, adjuvants, diluents, carriers and pharmaceutically active agents.
